(19) United States
(12) Patent Application Publication
(10) Pub. No.: US 2026/0108442 A1
(43) Pub. Date: Apr. 23, 2026

(54) COMPOUND FOR USE IN PROTECTING SKIN

(71) Applicants: TDELTAS LIMITED, Thame (GB); GOVERNMENT OF THE USA, AS REPRESENTED BY THE SECRETARY, DEPARTMENT OF HEALTH AND HUMAN SERVICES, Rockville, MD (US)

(72) Inventors: Kieran Clarke, Oxford (GB); Richard Lewis Veech, Rockville, MD (US)

(21) Appl. No.: 19/289,781

(22) Filed: Aug. 4, 2025

Related U.S. Application Data
(63) Continuation of application No. 17/032,616, filed on Sep. 25, 2020, now Pat. No. 12,377,032.
Application No. 17/032,616 is a continuation of application No. 14/774,856, now Pat. No. 10,821,062.

(30) Foreign Application Priority Data
Mar. 12, 2013 (GB) 1304467.2

(51) Int. Cl. A61K 8/37; A61G 17/00; A61K 8/86; A61K 9/00; A61K 9/06; A61K 31/22; A61K 31/357; A61K 47/10; A61Q 17/00; A61Q 17/04; A61Q 19/00; A61Q 19/08

(52) U.S. Cl. CPC A61K 8/375; A61G 17/00; A61K 8/86; A61K 9/0014; A61K 9/06; A61K 31/22; A61K 31/357; A61K 47/10; A61Q 17/00; A61Q 17/04; A61Q 19/00; A61Q 19/08

(57) ABSTRACT

A method of protecting animal tissue particularly skin, from damage caused by radiation exposure, by contacting the tissue with a ketone ester, a method of protecting skin, reducing the deterioration of skin or maintaining or improving the properties of skin by applying topically to the skin a ketone body comprising (R)-3-hydroxybutyrate moieties is disclosed. A ketone body comprising (R)-3-hydroxybutyrate moieties, especially enantiomerically enriched R-1,3-hydroxybutyl-(R)-3-hydroxybutyrate, for such uses is also provided.

### CROSS-REFERENCE TO RELATED APPLICATIONS

This application is a continuation of U.S. patent application Ser. No. 17/032,616, filed Sep. 25, 2020, which is a continuation of U.S. patent application Ser. No. 14/774,856, filed Sep. 11, 2015, now U.S. Pat. No. 10,821,062, which is a national phase entry under 35 USC § 371 of International Application No. PCT/EP2013/069189, filed Sep. 16, 2013, which claims priority to United Kingdom Application No. 1304467.2, filed Mar. 12, 2013. The entire contents of these applications are incorporated herein by reference in their entireties.

This invention relates to a compound for use in protecting skin, a method for preventing or reducing the risk of damage to skin and a composition for topical use to protect skin, to reduce the deterioration of skin due to ageing or due to the harmful effects of radiation or to maintain or to improve its properties. In particular, the invention relates to a ketone monoester for such uses and to a topical composition comprising the ketone ester.

It is known that radiation damages biological tissues and cells, for example skin. Treatment of skin to reduce the harmful effects of radiation, particularly ultra violet (UV) radiation, or deterioration due to ageing is well known.

Reducing the rate of deterioration or maintaining or improving skin properties, for example the appearance and feel of skin, has received a great deal of research effort and topical compositions for treating skin have been known since the earliest times. Dry skin also presents problems for many people. Personal care products such as skin creams and lotions, shampoos, conditioners, toilette bars, shower gels and antiperspirant and deodorants are typically normally formulated with at least one material to address dry skin. Symptoms such as itching, flaking and a visually displeasing dermal appearance can all to some extent be ameliorated. A wide range of products have been developed to address these problems and include occlusives such as petrolatum or silicone oils which serve to inhibit loss of natural moisture. Occlusives form a barrier between the epidermis and the environment. Keratolytic agents have also been employed to enhance rate of dermal exfoliation. Alpha-hydroxy acids are the most common agents for achieving exfoliation. A further approach is in the topical application of humectants and hydroxylated monomeric and polymeric organic substances are typically used for this purpose. Glycerin (glycerol) is well known for this use. Conventional topical applications typically deal primarily with the dead surface layers of the skin and may provide benefits upon application by the user, providing the user with a positive experience of ameliorating a skin condition or the effect of ageing.

It is also known to treat skin using ingestible ingredients to provide improvements in skin appearance and texture. These ingredients act by accessing the living interior of the skin. Examples of ingestible ingredients for treating skin include dietary fish oil and carotenoids such as lycopene and β-carotene which are known to offer protection against UVR-induced erythema upon ingestion. Vitamins E & C when taken orally in combination have also been shown to provide protection against UVR-induced erythema. Ingestible treatments may be perceived as providing a medical effect and may not provide the user with an immediate sense of improvement as compared to topical compositions.

There remains a need for compositions that can provide beneficial effects on skin including one or more of providing hydration, an anti-ageing effect, improved visual dermal appearance, reduced dryness, treating sunburn and the like and treating medical conditions for example eczema and psoriasis or other problems described above.

Ketone bodies and ketone body esters are known to reduce the levels of free fatty acids circulating in the plasma of a subject for example as disclosed in WO2004/105742 and ingestion of ketone bodies may lead to various clinical benefits. They are known for a treating a range of medical conditions including an enhancement of cognitive performance, treatment of cardiovascular conditions, diabetes and treatment of mitochondrial dysfunction disorders and in treating muscle fatigue and impairment and are also known to provide advantageous effects as a food product, nutritional supplement or supplement or as a nutraceutical for example in rehydration.

WO2004/108740 discloses a wide range of compounds and compositions containing (R)-3-hydroxybutyrate derivatives, with emphasis on oligomers of hydroxybutyrates, effective for elevating blood concentrations of ketone bodies. Ketone bodies are produced when fatty acids levels are raised in the body and are metabolised by the body for energy. In known applications, ketone bodies have been administered enterally or parenterally to a subject.

A wide range of ketone bodies containing R-3-hydroxybutyrate are known including oligomers, esters of oligomers, salts, the acid form, esters of the acid with mono, di or trihydric alcohols, However ketone bodies have not to date been employed topically for preventing or reducing the risk of damage to skin, to protect skin, to reduce the deterioration of skin due to ageing or due to the harmful effects of radiation or to maintain or to improve its properties.

Dermal fibroblasts are responsible for generating connective tissue and allowing the skin to recover from injury. Human dermal fibroblasts (HDF) in culture rely primarily upon glucose to fuel both aerobic and anaerobic metabolism. We have now found that a specific ketone body provides beneficial effects on skin and protects dermal fibroblasts from radiation, particularly UV radiation when applied topically. The ketone may also provides beneficial effects upon topical application to gums and soft tissue. The user may also experience positive psychological effects associated with the improved properties being the result of the process of the user themselves applying the ketone ester topically.

### BRIEF DESCRIPTION OF THE DRAWINGS

FIG. 1 is a graph depicting the percentage of live and dead human dermal fibroblasts following an incubation period under control conditions or in the presence of R-3-hydroxybutyl-(R)-3-hydroxybutyrate.

FIG. 2 is a graph depicting the effect of UV radiation on cell survival of human dermal fibroblasts that were pre-incubated or after-treated with R-3-hydroxybutyl-(R)-3-hydroxybutyrate.

### DETAILED DESCRIPTION OF THE INVENTION

The invention provides in a first aspect a ketone body comprising (R)-3-hydroxybutyrate moieties for topical use in protecting skin, reducing the deterioration of skin or maintaining or improving the properties of skin.

The invention provides in a second aspect a ketone body comprising (R)-3-hydroxybutyrate moieties for use in a method for protecting skin, reducing the deterioration of skin or maintaining or improving the properties of skin which comprises applying topically to the skin the said ketone body.

In a further aspect, the invention provides a method of protecting skin, reducing the deterioration of skin or maintaining or improving the properties of skin comprising applying topically to the skin a ketone body comprising (R)-3-hydroxybutyrate moieties.

The invention also provides in a further aspect for use a ketone body comprising (R)-3-hydroxybutyrate moieties as a topical agent for protecting skin, reducing the deterioration of skin or maintaining or improving the properties of skin.

Skin may deteriorate for many reasons including ageing or due to the harmful effects of radiation. The ketone ester of the invention may reduce the rate of such deterioration. The ketone ester comprising (R)-3-hydroxybutyrate moieties is especially beneficial in protecting human dermal fibroblasts from radiation, for example UV-B radiation. The ketone body may be applied at any time to the skin, before, during or after exposure to radiation.

Properties of skin which may be maintained or improved include one or more of providing hydration, an anti-ageing effect, improved visual dermal appearance, reduced dryness, treating sunburn and the like and treating medical conditions for example eczema and psoriasis.

In a further aspect, the invention provides a topical composition comprising (R)-3-hydroxybutyrate moieties and an excipient for topical application. The composition is suitable for topical use in one or more of protecting skin, reducing the deterioration of skin due to ageing or due to the harmful effects of radiation and maintaining or improving properties of skin.

Preferably, the excipient comprises a cosmetically acceptable carrier.

The invention also provides a method for improving skin properties comprising topically applying to the skin a composition according to the invention.

In a further aspect, the invention provides a method of treatment of skin comprising applying a composition to the skin, the composition comprising (R)-3-hydroxybutyrate moieties and an excipient for topical application to aid spreading across the skin or absorption into the skin of the (R)-3-hydroxybutyrate moieties.

Advantageously, the ketone body and composition according to the invention provides beneficial effects on skin properties upon topical application and a desirable user experience. Suitably, the composition is readily spreadable over skin and absorbable into skin but preferably does not pass through skin.

The ketone body and composition of the invention may also beneficially be applied topically to the gums or soft tissue to protect the same.

Without wishing to be bound by any theory, it is believed that the ketone bodies in the composition are metabolised to produce ATP for energy and nourishment to cells. Conventional skin treatments applied topically employ fats or the like to provide a physical effect or to prevent water evaporating, so retaining moisture in the dermis. Further, it is believed that conventional topical compositions for improving skin properties act in a physical way utilising bulk properties of the composition, for example by applying oils or retaining moisture. However the inventor has found that by employing the ketone ester which acts at a cellular level and applying the ester topically provides a combination of desirable user experience and improvement in skin properties.

The invention also provides a method of achieving an anti-ageing effect in the skin of a human or non-human mammal (preferably a human), which comprises applying to the skin of the human or non-human mammal an amount of a ketone body or a composition according to the invention which is effective to achieve said anti-ageing effect.

As used herein, the term “ketone”, “ketone body” or “ketone bodies” means a compound or species which is a ketone or a ketone body precursor, that is, a compound or species which is a precursor to a ketone and which may be converted or metabolised to a ketone. In a preferred embodiment the ketone body comprises a ketone body ester or a partial ester of a ketone body.

Any ketone body or ketone body ester containing (R)-3-hydroxybutyrate moieties may be employed in the invention. Preferably, the ketone body comprises a ketone monoester. Examples of suitable ketone bodies or compounds which provide (R)-3-hydroxybutyrate moieties in situ include oligomers of hydroxybutyrates, triolide, acetoacetate and esters thereof and any precursors of beta hydroxybutyrate which are metabolisable in skin cells.

We have surprisingly found that R-3-hydroxybutyl-(R)-3-hydroxybutyrate, a ketone monoester are metabolised more effectively than other forms of hydroxybutyrate, particularly oligomers.

According to a further aspect of the invention, there is provided a ketone body comprising R-3-hydroxybutyl-(R)-3-hydroxybutyrate for topical use in protecting skin reducing the deterioration of skin or maintaining or improving the properties of skin.

Preferably, the ketone body comprises enantiomerically enriched R-1,3-hydroxybutyl-(R)-3-hydroxybutyrate. R-1,3-hydroxybutyl-(R)-3-hydroxybutyrate monoester may be employed in combination with other ketone bodies or ketone body precursors, for example acetoacetate. The R-1,3-hydroxybutyl-(R)-3-hydroxybutyrate monoester may be present in an amount less than any other such ketone bodies but preferably is present in an amount more than any such other ketone body. In an especially preferred embodiment R-1,3-hydroxybutyl-(R)-3-hydroxybutyrate monoester is the only ketone body or ketone body precursor present in the composition of the invention.

The level of ketone body or a ketone body ester in the composition suitably comprises at least 1% by weight of ketone body more preferably at least 10% by weight and up to 95% by weight of the composition. Whilst a level of 15 to 30% by weight of the composition may be suitable, a composition comprising from 30 to 95%, especially 50 to 95% by weight of the composition may be preferred depending on the skin condition being treated.

A composition of the invention may further comprise a medium chain triglyceride (MCT) and, optionally, their associated fatty acids. MCTs comprise fatty acids with a chain length of between 5 and 12 carbon atoms. It is known that a diet rich in MCT increases blood ketone levels. Suitable medium chain triglycerides are represented by the following formula CH₂R₁—CH₂R₂—CH₂R₃ wherein R1, R2 and R3 are fatty acids having 5 to 12 carbon atoms. Preferably, MCTs wherein R1, R2, and R3 are fatty acids containing a six-carbon backbone (tri-C6:0) are employed.

Where an MCT is employed, suitably the composition of the invention comprises i) a ketone body, preferably a ketone monoester, more preferably a D-p-hydroxybutyrate monoester, ii) a MCT, preferably tri-C6:0 MCT and iii) a cosmetically acceptable carrier.

The composition of the invention may also comprise L-carnitine or a derivative of L-carnitine. Examples of derivatives of L-carnitine include decanoylcamitine, hexanoylcarnitine, caproylcarnitine, lauroylcarnitine, octanoylcarnitine, stearoylcarnitine, myristoylcarnitine, acetyl-L-carnitine, O-Acetyl-L-carnitine, and palmitoyl-L-carnitine. Where a carnitine is employed, suitably the composition of the invention comprises i) a ketone body, preferably a ketone monoester, more preferably a D-β-hydroxybutyrate monoester and ii) L-carnitine or a derivative of L-carnitine.

Where MCT and L-carnitine or its derivative is employed, suitably the MCT is emulsified with the carnitine. Preferably 10 to 500 g of emulsified MCT is combined with 10 to 2000 mg of carnitine for example 50 g MCT (95% triC8:0) emulsified with 50 g of mono- and di-glycerides combined with 500 mg of L-carnitine.

The MCT may be present in a greater amount than the ketone body but preferably the level of ketone body is greater than the level of the MCT.

The composition may be in solid form or in liquid form for example a suspension, dispersion and emulsion, or other forms known for topical application, for example a gel. Where the composition is solid, it is suitably made up into a topically applicable form prior to use, for example by dilution with water to form a paste, lotion or the like. Preferred forms of the composition include lotions, creams, roll-on formulations, sticks, mousses, aerosol and non-aerosol sprays and fabric (e.g. non-woven textile)-applied formulations.

The composition of the invention may be any substance applied to a human body for improving skin properties including one or more of anti-ageing effect, reduced wrinkles, other aspects of skin appearance, cleansing, odour control or general aesthetics. Non-limiting examples of suitable compositions include leave-on skin lotions and creams, shower gels, toilet bars, antiperspirants, deodorants, dental products, shave creams, depilatories, lipsticks, foundations, mascara, sunless tanners and sunscreen lotions.

The cosmetically acceptable carrier may be any known such carriers employed alone or in combination with other carriers. Amounts of the carrier may range from 1 to 99.9%, preferably from 70 to 95%, optimally from 80 to 90% by weight of the composition. Among the useful carriers are water, emollients, fatty acids, fatty alcohols, thickeners and combinations thereof. The carrier may be aqueous, anhydrous or an emulsion. Preferably the compositions are aqueous, especially water and oil emulsions of the W/O or O/W or triplex W/O/W variety. Water when present may be in amounts ranging from 5 to 95%, preferably from 20 to 70%, optimally from 35 to 60% by weight of the composition.

Emollient materials may serve as cosmetically acceptable carriers. These may be in the form of silicone oils, natural or synthetic esters and hydrocarbons. Amounts of the emollients may range anywhere from 0.1 to 95%, preferably between 1 and 50% by weight of the composition.

Silicone oils may be divided into the volatile and nonvolatile variety. The term “volatile” as used herein refers to those materials which have a measurable vapour pressure at ambient temperature. Volatile silicone oils are preferably chosen from cyclic (cyclomethicone) or linear polydimethylsiloxanes containing from 3 to 9, preferably from 4 to 5, silicon atoms.

Non-volatile silicone oils useful as an emollient material include polyalkyl siloxanes, polyalkylaryl siloxanes and polyether siloxane copolymers. The essentially non-volatile polyalkyl siloxanes useful herein include, for example, polydimethyl siloxanes with viscosities of from 5×10-6 to 0.1 m2/s at 25° C. Among the preferred non-volatile emollients useful in the present compositions are the polydimethyl siloxanes having viscosities from 1×10-5 to 4×10-4 m2/s at 25° C.

Another class of non-volatile silicones are emulsifying and non-emulsifying silicone elastomers. Representative of this category is dimethicone/vinyl dimethicone crosspolymer available as Dow Corning 9040, General Electric SFE 839, and Shin-Etsu KSG-18. Silicone waxes such as Silwax WS-L (dimethicone copolyol laurate) may also be useful.

Suitable ester emollients include:
a) Alkyl esters of saturated fatty acids having 10 to 24 carbon atoms. Examples thereof include behenyl neopentanoate, isononyl isonanonoate, isopropyl myristate and octyl stearate.
b) Ether-esters such as fatty acid esters of ethoxylated saturated fatty alcohols.
c) Polyhydric alcohol esters such as ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory. Particularly useful are pentaerythritol, trimethylolpropane and neopentyl glycol esters of C1-C30 alcohols.
d) Wax esters such as beeswax, spermaceti wax and tribehenin wax.
e) Sugar ester of fatty acids such as sucrose polybehenate and sucrose polycottonseedate.

Natural ester emollients principally are based upon mono-, di- and tri-glycerides. Representative glycerides include sunflower seed oil, cottonseed oil, borage oil, borage seed oil, primrose oil, castor and hydrogenated castor oils, rice bran oil, soybean, oil, olive oil, safflower oil, shea butter, jojoba oil and combinations thereof. Animal derived emollients are represented by lanolin oil and lanolin derivatives. Amounts of the natural esters may range from 0.1 to 20% by weight of the compositions.

Hydrocarbons which are suitable cosmetically acceptable carriers include petrolatum, mineral oil, C11-C13 isoparaffins, polybutenes, and especially isohexadecane, available commercially as Permethyl 101A from Presperse Inc.

Fatty acids having from 10 to 30 carbon atoms may also be suitable as a cosmetically acceptable carriers. Examples of this category include pelargonic, lauric, myristic, palmitic, stearic, isostearic, oleic, linoleic, linolenic, hydroxystearic and behenic acids.

Fatty alcohols having from 10 to 30 carbon atoms may also be suitable as a cosmetically acceptable carrier. Examples of this category include stearyl alcohol, lauryl alcohol, myristyl alcohol, oleyl alcohol and cetyl alcohol.

Thickeners can be utilized as part of the cosmetically acceptable carrier of compositions according to the present invention. Typical thickeners include crosslinked acrylates (e.g. Carbopol 982®), hydrophobically-modified acrylates (e.g. Carbopol 1382®), polyacrylamides (e.g. Sepigel 305®), acryloylmethylpropane sulfonic acid/salt polymers and copolymers (e.g. Aristoflex HMB® and AVC®), cellulosic derivatives and natural gums. Suitable cellulosic derivatives include sodium carboxymethylcellulose, hydroxypropyl methocellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Inorganics may also be utilized as thickeners, particularly clays such as bentonites and hectorites, fumed silicas, talc, calcium carbonate and silicates such as magnesium aluminum silicate (Veegum®). Amounts of the thickener may range from 0.0001 to 10%, usually from 0.001 to 1%, optimally from 0.01 to 0.5% by weight of the composition.

Adjunct humectants may be employed in the present invention. These are generally polyhydric alcohol-type materials. Typical polyhydric alcohols include glycerol, propylene glycol, dipropylene glycol, polypropylene glycol, polyethylene glycol, sorbitol, hydroxypropyl sorbitol, hexylene glycol, 1,3-butylene glycol, isoprene glycol, 1,2,6-hexanetriol, ethoxylated glycerol, propoxylated glycerol and mixtures thereof. The amount of adjunct humectant may range anywhere from 0.5 to 50%, preferably between 1 and 15% by weight of the composition.

Surfactants may also be present in compositions of the present invention. Total concentration of the surfactant when present may range from 0.1 to 90%, preferably from 1 to 40%, optimally from 1 to 20% by weight of the composition, and is highly dependent upon the type of personal care product. The surfactant may be selected from the group consisting of anionic, nonionic, cationic and amphoteric actives. Particularly preferred nonionic surfactants are those with a C10-C20 fatty alcohol or acid hydrophobe condensed with from 2 to 100 moles of ethylene oxide or propylene oxide per mole of hydrophobe; C2-C10 alkyl phenols condensed with from 2 to 20 moles of alkylene oxide; mono- and di-fatty acid esters of ethylene glycol; fatty acid monoglyceride; sorbitan, mono- and di-C8-C20 fatty acids; and polyoxyethylene sorbitan as well as combinations thereof. Alkyl polyglycosides and saccharide fatty amides (e.g. methyl gluconamides) and trialkylamine oxides are also suitable nonionic surfactants.

Preferred anionic surfactants include soap, alkyl ether sulfates and sulfonates, alkyl sulfates and sulfonates, alkylbenzene sulfonates, alkyl and dialkyl sulfosuccinates, C8-C20 acyl isethionates, C6-C20 alkyl ether phosphates, C8-C20 sarcosinates, C8-C20 acyl lactylates, sulfoacetates and combinations thereof.

Useful amphoteric surfactants include cocoamidopropyl betaine, C12-C20 trialkyl betaines, sodium lauroamphoacetate, and sodium laurodiamphoacetate.

Sunscreen agents may also be included in compositions of the present invention. Particularly preferred are such materials as ethylhexyl p-methoxycinnamate (Parsol MCX®), avobenzene (Parsol 1789®) and benzophenone-3 (also known as oxybenzone). Inorganic sunscreen actives may be employed such as microfine titanium dioxide and zinc oxide. Amounts of the sunscreen agents when present may generally range from 0.1 to 30%, preferably from 2 to 20%, optimally from 4 to 10% by weight of the composition.

Antiperspirants and deodorant compositions of the present invention ordinarily will contain astringent actives. Examples include aluminum chloride, aluminum chlorhydrex, aluminum-zirconium chlorhydrex glycine, aluminum sulfate, zinc sulfate, zirconium and aluminum chlorohydroglycinate, zirconium hydroxychloride, zirconium and aluminum lactate, zinc phenolsulfonate and combinations thereof. Amounts of the astringents may range anywhere from 0.5 to 50% by weight of the composition.

Preservatives can desirably be incorporated into the personal care compositions of this invention to protect against the growth of potentially harmful microorganisms. Particularly preferred preservatives are phenoxyethanol, methyl paraben, propyl paraben, imidazolidinyl urea, dimethyloldimethylhydantoin, ethylenediaminetetraacetic acid salts (EDTA), sodium dehydroacetate, methylchloroisothiazolinone, methylisothiazolinone, iodopropynbutylcarbamate and benzyl alcohol. The preservatives should be selected having regard for the use of the composition and possible incompatibilities between the preservatives and other ingredients. Preservatives are preferably employed in amounts ranging from 0.0001% to 2% by weight of the composition.

Compositions of the present invention may include vitamins. Illustrative vitamins are vitamin A (retinol), vitamin B2, vitamin B3 (niacinamide), vitamin B6, vitamin C, vitamin E, folic acid and biotin. Derivatives of the vitamins may also be employed. For instance, vitamin C derivatives include ascorbyl tetraisopalmitate, magnesium ascorbyl phosphate and ascorbyl glycoside. Derivatives of vitamin E include tocopheryl acetate, tocopheryl palmitate and tocopheryl linoleate. DL-lianthenol and derivatives may also be employed. Total amount of vitamins when present in compositions according to the present invention may range from 0.001 to 10%, preferably from 0.01% to 1%, optimally from 0.1 to 0.5% by weight-of the composition.

An enzyme may be present as desired, for example amylases, oxidases, proteases, lipases and combinations thereof. Particularly preferred is superoxide dismutase, commercially available as Biocell SOD from the Brooks Company, USA.

Skin lightening compounds may be included in the compositions of the invention. Illustrative substances are placental extract, lactic acid, niacinamide, arbutin, kojic acid, ferulic acid, resorcinol and derivatives including 4-substituted resorcinols and combinations thereof. Amounts of these agents may range from 0.1 to 10%, preferably from 0.5 to 2% by weight of the composition.

A variety of herbal extracts may optionally be included in compositions of this invention. The extracts may either be water soluble or water-insoluble carried in a solvent which respectively is hydrophilic or hydrophobic. Water and ethanol are the preferred extract solvents. Illustrative extracts include those from green tea, chamomile, liquorice, aloe vera, grape seed, citrus unshui, willow bark, sage, thyme and rosemary.

Further carrier components may be included such as lipoic acid, retinoxytrimethylsilane (available from Clariant Corp. under the Silcare IM-75 trademark), dehydroepiandrosterone (DHEA) and combinations thereof. Ceramides (including Ceramide 1, Ceramide 3, Ceramide 3B and Ceramide 6) as well as pseudoceramides may also be useful. Amounts of these materials may range from 0.000001 to 10%, preferably from 0.0001 to 1% by weight of the composition.

Colorants, opacifiers and abrasives may also be included in compositions of the present invention. Each of these substances may range from 0.05 to 5%, preferably between 0.1 and 3% by weight of the composition.

The compositions of the present invention can also be, optionally, incorporated into an insoluble substrate for application to the skin such as in the form of a treated wipe.

Another aspect this invention is the inclusion of instructions attached to or otherwise associated with the packaging in which the ketone body or composition of the invention is supplied. The instructions indicate to a consumer topical use of the ketone body or composition on skin, hair or oral mucosae. Packaging itself will usually be printed with the instructions but sometimes a separate written insert within the package may serve to provide the instructions. Typical language includes phrases such as “apply a thin layer to the underarm”, “apply regularly to hands”, “cleanse skin” and “pump a small amount onto the palm of your hand”.

Ketone bodies and compositions of the invention are suitable for improving skin properties and especially in providing an anti-ageing effect. By the term “anti-ageing”, we mean that the skin may appear less wrinkled (i.e., there is an anti-wrinkling effect on wrinkles and/or fine lines, including a reduction in wrinkle depth) and that the composition may impart one or more further benefits for the skin selected from: reduced dryness; increased firmness; increased elasticity; increased smoothness; clearer skin; fewer spots, pimples and blemishes (including acne); clearer skin; less sensitive skin; and generally healthier skin.

Ketone bodies and compositions of the invention may exhibit the anti-ageing effect by improving skin cell nutrition or for example by increasing collagen synthesis in the skin and compositions of the invention may be used to increase collagen synthesis (as part of, or separately from, the anti-ageing effect); preferably collagen synthesis is increased by at least 10%, more preferably at least 20% such as at least 25% by weight (e.g., as determined based on the weight of collagen synthesised, preferably over a 14 week period).

The skin may include the skin of the whole body, preferably the face, neck and/or hands. The skin may also include scalp skin with benefits for hair (including reduced ageing) and scalp itch or irritation.

The following examples are illustrative embodiments of this invention. All parts, percentages and proportions referred to herein and in the appended claims are by weight unless otherwise illustrated.

Various methods of measurement of characteristics of the skin are known and provide a means of determining by measurement or visually improvements in skin properties. Examples of such methods include:

### Measurement of Skin Hydration

Various methods for determining the hydration state of the stratum corneum have been summarized by Fluhr et al., Skin Res Technol 1999; 5:161-170. Briefly, the Corneometer (Courage & Khazaka) measures skin hydration through detection of epidermal capacitance. The probe is made of two finger-type metal plates close to each other, with a measurement depth of approximately 30 mm. The instrument determines the humidity level of the most external cutaneous layers of the stratum corneum. The action principle of the Corneometer® is based on the modification of the electrical capacities of the detector which is designed in the form of a condenser. The surface of the measurement head, in contact with the skin, modifies its electrical capacity according to the humidity level of the skin. An increase in the value measured by the corneometer is indicative of improved skin hydration.

### Measurement of Trans Epidermal Water Loss (TEWL)

An analysis of methods to measure TEWL has been performed by Wilson & Maibach, (1989) Transepidermal water loss, A review, In: Cutaneous Investigation in Health and Disease, Noninvasive Methods and Instrumentation (Leveque, J. L., ed.), pp. 113-130, Dekker, New York, NY. The cutaneous barrier acts as a regulator in skin water balance. When this is damaged, the water exchange regulation system becomes destabilised. This means that water migrates more easily to the outside environment, increasing Transepidermal Water Loss. The effectiveness of the cutaneous barrier decreases with age. However, if the condition of the cutaneous barrier improves, water loss decreases as the water exchange regulation mechanism recovers its balance. TransEpidermal Water Loss measurements can be performed with a Servomed “Evaporimeter” EP-3®. A probe made up of two captors is traversed by a flow of water vapour. The difference of the partial pressure is measured between the two captors. This value corresponds to the evaporation speed of a volatile substance (in this case, water). A reduction in TEWL is indicative of improved skin barrier properties

### Measurement of Skin Elasticity & Firmness

Measurements for skin elasticity and firmness are made with a cutometer and described in Escoffier et al, J Invest Dermatol, 93(3): 353-7. The measurement is done with an instrument which, using the vacuum principle, sucks up a defined area of skin surface and records it optically. Analysis of the recorded measurement curves makes it possible to determine the elastic and plastic characteristics of the skin. Young skin shows a high degree of elasticity and loses shape only gradually while regaining its original state after the end of the suction procedure. Skin which is young, healthy, supple and adequately moist will have a higher elasticity than an aged dry, rough skin. The cutometer therefore gives a set of measurements which allows us to quantify elastic characteristics. The technique consists of skin aspiration by a measurement probe. The skin is sucked into the orifice of the probe by negative pressure created within the device. The depth to which the skin penetrates into the probe is measured by a non-contact optical measurement system. This system consists of a light source and light receptor, as well as two prisms facing each other, which project the light from transmitter to receptor. Light intensity varies with penetration depth of the skin. The resistance of the skin to be sucked up gives an indication of the firmness of the skin and the ability to return to its original position gives an indication of the elasticity of the skin. A curve is displayed at the end of each measurement which allows several calculations to be made corresponding to skin mechanical properties.

### Analysis of Fine Lines, Wrinkles & Skin Smoothness

Skin roughness and wrinkling can be assessed using replicas and skin profilometry as described by Cook, J Soc Cosmet Chem, 1980; 31:339-359. A silicon rubber material such as Silflo is prepared and applied to the test area. Once set it is removed and analysed using optical profilometry. With this measurement method, a parallel stripe pattern is projected onto the skin surface and depicted on the CCD chip of a camera. The 3D measurement effect is achieved by the fact that minute evaluation differences on the skin surface deflect the parallel projection stripes and that these deflections constitute a qualitative and quantitative measurement of the skin profile. The skin profiles are recorded by the CCD camera, digitised, and transferred to the measurement and evaluation computer for qualitative evaluation.

### Example 1—Composition of the Invention

A composition of the present invention in the form of a cosmetic lotion for topical use is outlined in Table I.

TABLE I

| | |
|---|---|
| INGREDIENT | WEIGHT % |

PHASE A

| | |
|---|---|
| Water | Balance |
| Disodium EDTA | 0.05 |
| Methyl paraben | 0.15 |
| Magnesium aluminum silicate | 0.60 |
| Triethanolamine | 1.20 |
| a D-betahydroxybutyrate butanediol monoester | 1.00 |

PHASE B

| | |
|---|---|
| Xanthan gum | 0.20 |
| Natrosol ® 250 HHR (ethyl cellulose) | 0.50 |
| Butylene glycol | 3.00 |
| Glycerin | 2.00 |

PHASE C

| | |
|---|---|
| Sodium stearoyl lactylate | 0.10 |
| Glycerol monostearate | 1.50 |
| Stearyl alcohol | 1.50 |
| Isostearyl palmitate | 3.00 |
| Silicone fluid | 1.00 |
| Cholesterol | 0.25 |
| Sorbitan stearate | 1.00 |
| Butylated hydroxy toluene | 0.05 |
| Vitamin E acetate | 0.01 |
| PEG-100 stearate | 2.00 |
| Stearic acid | 3.00 |
| Propyl paraben | 0.10 |
| Parsol MCX ® | 2.00 |
| Caprylic/capric triglyceride | 0.50 |
| Hydroxycaprylic acid | 0.01 |
| C12-15 alkyl octanoate | 3.00 |

PHASE D

| | |
|---|---|
| Vitamin A palmitate | 0.10 |
| Bisabolol | 0.01 |
| Vitamin A acetate | 0.01 |
| Fragrance | 0.03 |
| Retinol SOC | 0.02 |
| Conjugated linoleic acid | 0.50 |

The composition is suitably prepared by adding the phases in sequence and homogenising the mixture.

### Example 2

A composition of the present invention in the form of a skin cream for topical use is shown in Table II.

TABLE II

| INGREDIENT | WEIGHT % |
|---|---|
| Glycerin | 6.93 |
| Niacinamide | 5.00 |
| D-betahydroxybutyrate butanediol monoester | 5.00 |
| Permethyl ™ 101A1 | 3.00 |
| Sepigel ™ 3052 | 2.50 |
| Q2-14033 | 2.00 |
| Linseed oil | 1.33 |
| Arlatone ™ 21214 | 1.00 |
| Cetyl alcohol CO-1695 | 0.72 |
| SEFA cottonate5 | 0.67 |
| Tocopherol acetate | 0.50 |
| Panthenol | 0.50 |
| Stearyl alcohol | 0.48 |
| Titanium dioxide | 0.40 |
| Disodium EDTA | 0.10 |
| Glydant ™ Plus6 | 0.10 |
| PEG-10 stearate | 0.10 |
| Stearic acid | 0.10 |
| Purified water | Balance |

1Isohexadecane (Presperse Inc., South Plainfield, NJ);
3Polyacrylamide (and) C13-14 isoparaffin(and) laureth-7 (Seppic Corporation, Fairfield, NJ);
3Dimethicone (and) dimethiconol (Dow Corning Corp. Midland, MI);
4Sorbitan monostearate and sucrococoate (ICI Americas Inc., Wilmington DE);
5Sucrose ester of fatty acid;
6DMDM Hydantoin (and) iodopropynyl butylcarbamate (Lonza Inc., Fairlawn, NJ).

### Example 3

A cosmetic composition of the present invention for topical use is shown in Table III.

TABLE III

| INGREDIENT | WEIGHT % |
|---|---|
| Polysilicone-11 | 29 |
| Cyclomethicone | 59 |
| Petrolatum | 11 |
| a D-betahydroxybutyrate butanediol monoester. | 0.2 |
| Dimethicone copolyol | 0.5 |
| Sunflowerseed oil | 0.3 |

### Example 4

A disposable, single use personal care towelette product is described according to the present invention. A 70/30 polyester/rayon non-woven towelette is prepared with a weight of 1.8 grams and dimensions of 15 cm by 20 cm. Onto this towelette is impregnated a composition with a hydroxybutyrate ester as shown in Table IV below.

TABLE IV

| INGREDIENT | WEIGHT % |
|---|---|
| D-betahydroxybutyrate butanediol monoester | 7.50 |
| Glycerin | 2.00 |
| Hexylene glycol | 2.00 |
| Disodium capryl amphodiacetate | 1.00 |
| Gluconolactone | 0.90 |
| Silicone microemulsion | 0.85 |
| Witch hazel | 0.50 |
| PEG-40 hydrogenated-castor oil | 0.50 |
| Fragrance | 0.20 |
| Vitamin E acetate | 0.001 |
| Water | Balance |

### Example 5

D-betahydroxybutyrate butanediol monoester was used neat in this experiment. The hands of the user were visually inspected prior to application of the D-betahydroxybutyrate butanediol monoester. The hands had a dry appearance and feel. D-betahydroxybutyrate butanediol monoester was applied to the hands and massaged in to the skin thoroughly. The hands were inspected visually again after 30 minutes and were observed to have a visually noticeable improvement in properties including less dryness, less wrinkling and an improved feel.

### Example 6

In this example, the effects of R-3-hydroxybutyl-(R)-3-hydroxybutyrate on human dermal fibroblast growth were assessed.

Primary human dermal fibroblasts (HDF), with a seeding capacity of 2,500 cells/cm², from TCS Cellworks were grown in 96-well plates for 24, 48 and 72 hours in either human fibroblast (HF) basal medium (TCS Cellworks) or basal medium containing 4 mM of the ketone ester, deltaG. The basal medium was changed daily.

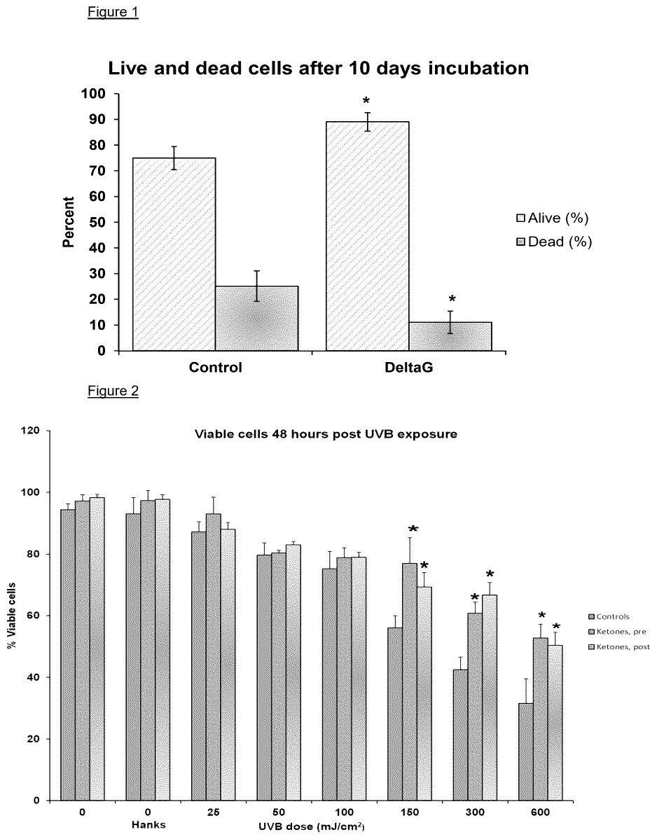

Cell viability was determined using a live and dead assay kit, with two colour fluorescence staining of live and dead cells using the probes, Calcein AM and EthD-III. After 10 days incubation, the numbers of live cells was significantly greater when grown in the presence of R-3-hydroxybutyl-(R)-3-hydroxybutyrate (89% vs. 75%, P<0.05) with fewer dead cells. The results are shown in FIG. 1. It can be concluded that the ketone ester according to the invention decreased human dermal fibroblast death.

### Example 7

Human dermal fibroblast cells were cultured in basal medium in 6-well plates at 50,000 cells per well. The cells were incubated in medium alone or with added 4 mM R-3-hydroxybutyl-(R)-3-hydroxybutyrate. After reaching 80-90% confluence, cells were washed with phosphate-buffered saline and the medium was replaced with a thin layer of Hanks buffer, (a balanced salt solution designed to use with cells in non-CO₂ atmospheric conditions). Cells were irradiated at doses of 25, 50, 100, 150, 300 and 600 mJ/cm² using a portable UVB source (302 nm) with a fluence rate of 0.9 mW/cm² at cell level. UV irradiation was performed in a fume hood to maintain sterility. After irradiation, cells were washed and incubated in medium alone or with added 4 mM R-3-hydroxybutyl-(R)-3-hydroxybutyrate (as they had been before irradiation). Another group of irradiated cells, which had initially been grown in basal medium without deltaG, was incubated with added 4 mM R-3-hydroxybutyl-(R)-3-hydroxybutyrate. Cell survival was determined by counting viable cells using Trypan blue exclusion at 48 hours after irradiation. The results are shown in FIG. 2

It was found that R-3-hydroxybutyl-(R)-3-hydroxybutyrate protected human dermal fibroblasts from UVB radiation (p<0.05) at doses between 150 and 600 mJ/cm² when given to the cells after radiation. Providing R-3-hydroxybutyl-(R)-3-hydroxybutyrate both before and after radiation exposure conferred no further benefit.

In summary, R-3-hydroxybutyl-(R)-3-hydroxybutyrate both increased human dermal fibroblast viability and protected dermal fibroblasts from UV damage.

1. (canceled)

2. A method of maintaining or improving the properties of skin comprising applying topically to the skin of a subject in need thereof an effective amount of a (R)-3-hydroxybutyrate moiety or a composition comprising a m (R)-3-hydroxybutyrate moiety and an excipient for topical application to aid spreading across the skin or absorption into the skin of the (R)-3-hydroxybutyrate moiety.

3-9. (canceled)

10. A topical composition comprising a (R)-3-hydroxybutyrate moiety and an excipient, wherein the topical composition is formulated for topical application to skin.

11. The topical composition of claim 10, wherein the excipient comprises a cosmetically acceptable carrier.

12. A method of protecting skin, reducing the deterioration of skin, or maintaining or improving the properties of skin, said method comprising administering an effective amount of the topical composition of claim 10 to a subject in need thereof.

13. The topical composition of claim 10, wherein the level of the (R)-3-hydroxybutyrate moiety in the composition is at least 1% by weight.

14. The topical composition of claim 10, wherein the excipient comprises polyethylene glycol.

15. The method of claim 2, wherein the method further provides protection of the skin of the subject.

16. The method of claim 2, wherein the method further provides a reduction in the deterioration of the skin of the subject or an improvement in the cosmetic appearance of the skin of the subject.

17. The topical composition of claim 10, wherein the (R)-3-hydroxybutyrate moiety is selected from an ester of (R)-3-hydroxybutyrate, an oligomer of (R)-3-hydroxybutyrate, triolide, acetoacetate and esters thereof, a salt of (R)-3-hydroxybutyrate; acetoacetyl oligomers, and any precursor of (R)-3-hydroxybutyrate which is capable of being metabolised in skin cells.

18. The method of claim 12, wherein the method provides maintenance or improvement in the properties of skin or a reduction in the deterioration of the skin due to ageing or due to the harmful effects of radiation.

19. A skin product in the form of a liquid, gel, cream, lotion, mousse, aerosol, shampoo, conditioner, toilet bar, antiperspirant, deodorant, depilatory lipstick, foundation product, mascara, sunless tanner or sunscreen lotion, wherein the skin product comprises a (R)-3-hydroxybutyrate moiety selected from an ester of (R)-3-hydroxybutyrate, an oligomer of (R)-3-hydroxybutyrate, triolide, acetoacetate and esters thereof, a salt of (R)-3-hydroxybutyrate; acetoacetyl oligomers, and any precursor of (R)-3-hydroxybutyrate which is capable of being metabolised in skin cells.

20. The skin product according to claim 17, further comprising at least one of a ceramide, a pseudo-ceramide, or a vitamin.

21. The skin product of claim 18, further comprising:
(i) a ceramide selected from the group consisting of Ceramide 1, Ceramide 3, Ceramide 3B and Ceramide 6; and/or
(ii) a vitamin selected from the group consisting of vitamin A (retinol), vitamin B2, vitamin B3 (niacinamide), vitamin B6, vitamin C, vitamin E, folic acid and biotin.

22. The skin product of claim 19, wherein the (R)-3-hydroxybutyrate moiety comprises 3-hydroxybutyl-(R)-3-hydroxybutyrate.

23. The skin product of claim 22, wherein the 3-hydroxybutyl-(R)-3-hydroxybutyrate is enantiomerically enriched R-1,3-hydroxybutyl-(R)-3-hydroxybutyrate.
